(19) United States
(12) Patent Application Publication
(10) Pub. No.: US 2026/0232863 A1
(43) Pub. Date: Aug. 13, 2026

(54) ION GENERATOR DEVICE

(71) Applicant: Air+, Inc., Rye, NY (US)

(72) Inventors: Lawrence T. Sunshine, Rye Brook, NY (US); Michael Robert, Farmington Hills, MI (US)

(21) Appl. No.: 19/533,045

(22) Filed: Feb. 6, 2026

Related U.S. Application Data
(60) Provisional application No. 63/755,850, filed on Feb. 7, 2025.

(51) Int. Cl. A61L 9/22; B01D 53/32; B03C 3/41; B03C 3/82

(52) U.S. Cl. CPC A61L 9/22; B01D 53/323; B03C 3/41; B03C 3/82; A61L 2209/12; A61L 2209/15; A61L 2209/16; B03C 2201/06

(57) ABSTRACT

Ion generator devices are provided. An ion generator device may comprise two ionizing elements respectively mounted in emitter pockets within the ion generator device. The ion generator device may have a wall extending between the ionizing elements, which is formed of a dielectric material and has a dielectric strength sufficient to maintain its dielectric property at operating voltages. The ion generator device may be mounted to a mounting target using magnets positioned in respective recesses in a mounting wall of the ion generator device. The ion generator device may have an ionization indicator light which indicates a status of the ion generator device. A wall of the ion generator device may have an indicator opening aligned with the ionization indicator light such that the ionization indicator light is viewable on an external surface.

### CROSS REFERENCE TO RELATED APPLICATIONS

This application claims the benefit of U.S. Provisional Application No. 63/755,850 filed on Feb. 7, 2025, which is incorporated by reference herein in its entirety.

### FIELD OF THE DISCLOSURE

This disclosure relates to bi-polar ion generator devices.

### BACKGROUND

Air flow systems typically comprise one or more elements which move and/or treat air. An air flow system may treat air by heating or cooling the air. The air circulating within an air flow system and the space served by the air flow system may be contaminated with foreign contaminants such as particulates, microorganisms and volatile organic compounds. An air flow system may also treat the air by neutralizing the contaminants (e.g., decontaminate).

Ionizers, also known as ion generator devices, may be used to decontaminate air circulating within an air flow system and the space served by the air flow system. Certain ionizers may include high voltage electrodes. In order to decontaminate the air circulating within the air flow system and the space served by the air flow system, one or more ion generator devices may need to be mounted within the air flow system such that the produced ions may flow into the air. However, depending on the application, the space in which to mount the ion generator device(s) may be small, causing a difficulty in mounting. For example, due to the small space, an installer may not be able to use a hand tool or power tool, such as a power screwdriver, to install mounting hardware to mount the ion generator device. Additionally, in certain applications, the installed position renders it nearly impossible to directly view the installed ion generator device. This makes it difficult to determine, after installation, whether the ion generator device is working as intended.

An ion generator device may produce positive ions, negative ions and both positive ions and negative ions. For example, an ion generator device may have a first electrode for producing positive ions and a second electrode for producing negative ions where the first electrode and second electrode are spaced apart. In order to produce the ions, a high voltage is applied to each electrode to produce a positive or negative charge, Q (Coulombs). This results in an electric field being generated by each electrode. The electric field, E (V/m) is proportional to the charge, Q multiplied by Coulomb's constant, k (8.9875×10^{9N}·m²*C⁻²) and divided by the distance from the electrode, r squared.

$$\vec{E} = \frac{k * Q}{{r}^{2}}$$

The high electric field strength between two oppositely charged electrodes is additive and may create an electric field strong enough to break down the dielectric strength of the air between the electrodes and cause the production of ozone.

### SUMMARY

Accordingly, disclosed is an ion generator device comprising a mounting wall, opposing sidewalls, first and second walls and a cover. The opposing sidewalls project from the mounting wall. The first wall projects from an edge of the mounting wall. The edge is an edge in a second direction orthogonal to a first direction. The opposing sidewalls are connected to the first wall. The second wall extends between the opposing sidewalls in the first direction. At least a portion of the mounting wall, the opposing sidewalls, the first wall and the second wall form a compartment for a circuit board of the ion generator device. The circuit board is connected to a first ionizing element and a second ionizing element configured to produce ions. The first ionizing element and the second ionizing element are mounted in respective emitter pockets. The cover extends between the opposing sidewalls in the first direction. The cover is configured to cover the compartment. The ion generator device further comprises a wall extending between the first ionizing element and the second ionizing element. The wall is formed of a dielectric material and has a dielectric strength sufficient to maintain its dielectric property at operating voltages.

In an aspect of the disclosure, the first ionizing element and the second ionizing element are mounted in the respective emitter pockets such that tips of the first ionizing element and the second ionizing element do not extend beyond the ion generator device. In an aspect of the disclosure, the ion generator device further comprises a first clip and a second clip configured to respectively hold the first ionizing element and the second ionizing element such that the first ionizing element and the second ionizing element are held in a middle of a respective emitter pocket.

In an aspect of the disclosure, the opposing sidewalls are offset from a first edge and a second edge of the mounting wall in a first direction. The mounting wall may have a first recess and a second recess. The first recess and the second recess may be configured to respectively receive a magnet for mounting the ion generator device to an external surface.

In an aspect of the disclosure, the first recess may extend both on a first side of one of the opposing sidewalls and a second side of the one of the opposing sidewalls in the first direction and the second recess may extend both on a first side of another opposing sidewall and a second side of the another opposing sidewall in the first direction.

In an aspect of the disclosure, the first recess and the second recess may be dimensioned to enable a magnetic field of a respective magnet to be a sufficient strength at the external surface to hold the ion generator device to the external surface. The edges of the first recess and the second recess may be chamfered, and the first recess and the second recess may extend in the first direction a greater distance than the respective magnet.

In an aspect of the disclosure, the ion generator device may be mounted using different mounting methods. For example, in an aspect of the disclosure, the mounting wall may have at least two openings configured to receive mounting hardware to attach the ion generator device to the external surface, respectively and/or the first wall which extends from the first edge to the second edge in the first direction has at least two openings configured to receive mounting hardware to attach the ion generator device to another external surface. The ion generator device may also be magnetically mounted.

In an aspect of the disclosure, the ion generator device may also comprise an ionization indicator light. A wall of the ion generator device may have an indicator opening aligned with the ionization indicator light. In an aspect, an interior surface of the wall with the indicator opening may have a bevel edge around the indicator opening. The ionization indicator light may be positioned within the bevel edge adjacent to the indicator opening.

In an aspect of the disclosure, the ionizing elements may be ionizing needles or ionizing brushes. The ionizing element may produce both positive and negative ions. In other aspects, one ionizing element may produce positive ions and the other may produce negative ions.

In an aspect of the disclosure, the length of the ion generator device in a third direction orthogonal to the first direction and the second direction may be equal to or less than about 1 in.

Also disclosed is an ion generator device comprising a mounting wall having a first recess and a second recess. The first recess and the second recess are configured to respectively receive a magnet for mounting the ion generator device to an external surface. The ion generator device also comprises opposing sidewalls projecting from the mounting wall. The opposing sidewalls are offset from a first edge and a second edge of the mounting wall in a first direction. The ion generator device also comprises a first wall projecting from a third edge of the mounting wall. The third edge is an edge in a second direction orthogonal to the first direction. The opposing sidewalls are connected to the first wall. The ion generator device also comprises a second wall extending between the opposing sidewalls. A portion of the mounting wall, the opposing sidewalls, the first wall and the second wall form a compartment for a circuit board of the ion generator device. The circuit board is connected to a first ionizing element and a second ionizing element configured to produce ions. The ion generator device also comprises a cover extending between the opposing sidewalls. The cover is configured to cover the compartment when attached.

In an aspect of the disclosure, each recess may be positioned on both sides of a respective sidewall in the first direction.

In an aspect of the disclosure, each recess may be dimensioned to enable a magnetic field of a respective magnet to be a sufficient strength at the external surface to hold the ion generator device to the external surface.

In an aspect of the disclosure, the edges of each recess may be chamfered. Each recess may also be oversized with respect to a respective magnet in both the first and second directions.

In an aspect of the disclosure, in addition to being mountable using the magnets, the ion generator device may be mounted using mounting hardware. For example, in an aspect of the disclosure, the mounting wall may have at least two openings configured to receive mounting hardware to attach the ion generator device to the external surface, respectively and/or the first wall which extends from the first edge to the second edge in the first direction may have at least two openings configured to receive mounting hardware to attach the ion generator device to another external surface.

Also disclosed is an ion generator device comprising an ionization indicator light and a wall with an indicator opening. This wall is externally facing. The indicator opening is aligned with the ionization indicator light such that the ionization indicator light is viewable on an external surface. The ion generator device also comprises a mounting wall, opposing sidewalls projecting from the mounting wall, first and second walls, and a cover. The first wall projects from an edge of the mounting wall. This edge is an edge in a second direction orthogonal to a first direction. The opposing sidewalls are connected to the first wall. The second wall extends between the opposing sidewalls in the first direction. At least a portion of the mounting wall, the opposing sidewalls, the first wall and the second wall form a compartment for a circuit board of the ion generator device. The circuit board is connected to a first ionizing element and a second ionizing element configured to produce ions.

In an aspect of the disclosure, the ionization indicator light may be a light emitting diode (LED) with a viewing angle of less than a predetermined angle.

In an aspect of the disclosure, the ion generator device has a protective film or lens is installed on or in the indicator opening.

In an aspect of the disclosure, an interior surface of the wall has a bevel edge around the indicator opening. The ionization indicator light may be positioned within the bevel edge adjacent to the indicator opening.

In an aspect of the disclosure, the indicator opening may have a diameter of about 3 mm.

### DESCRIPTION

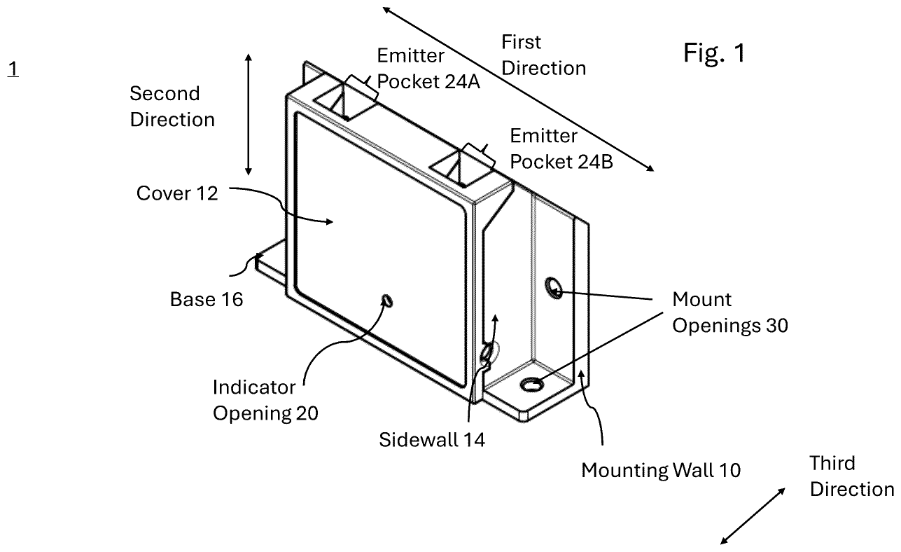

FIG. 1 illustrates an isometric view of an ion generator device in accordance with aspects of the disclosure;

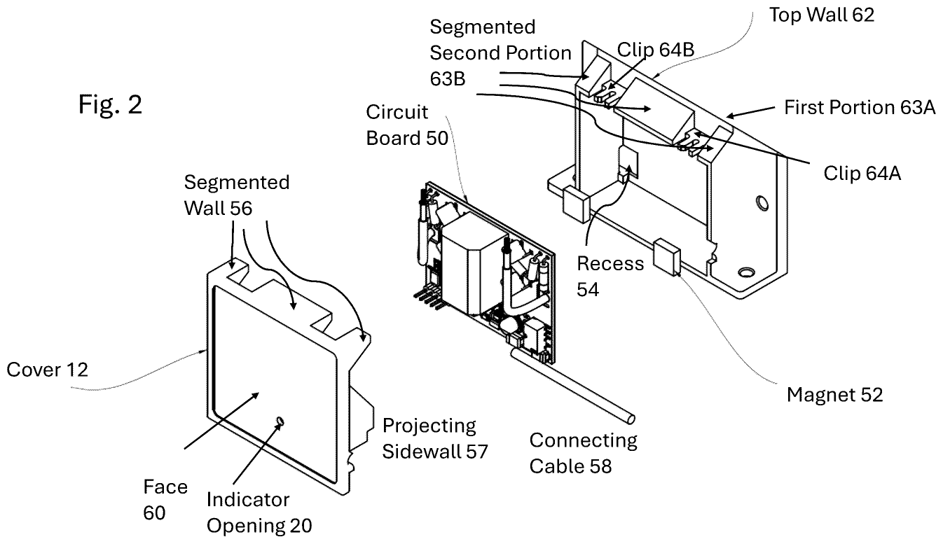

FIG. 2 illustrates an exploded view of the ion generator device shown in FIG. 1 in accordance with aspects of the disclosure;

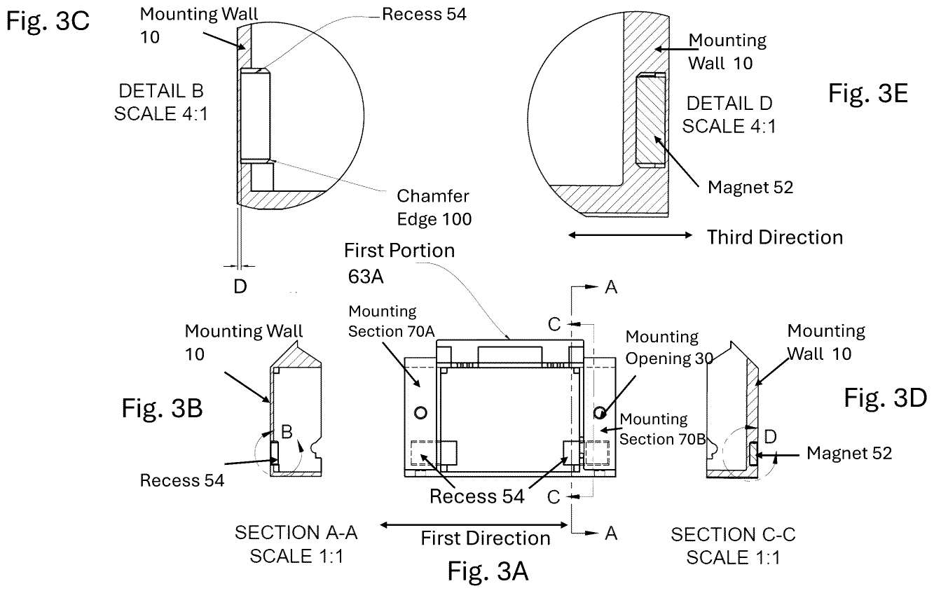

FIG. 3A illustrates a view of the ion generator device shown in FIG. 1 in accordance with aspects of the disclosure;

FIG. 3B illustrates a sectional view along the A-A line in FIG. 3A;

FIG. 3C illustrates a zoomed view of the area B in FIG. 3B;

FIG. 3D illustrates a sectional view along the C-C line in FIG. 3A;

FIG. 3E illustrates a zoomed view of the area D in FIG. 3D;

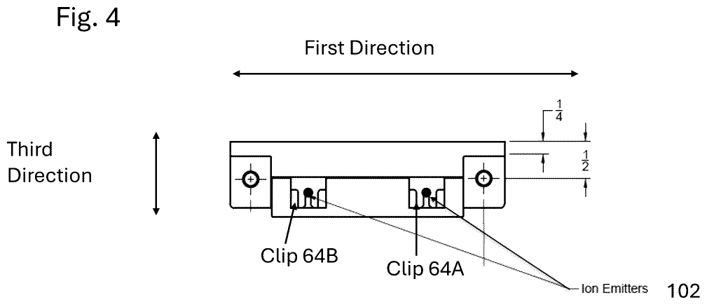

FIG. 4 illustrates a top view of the ion generator device shown in FIG. 1 in accordance with aspects of the disclosure;

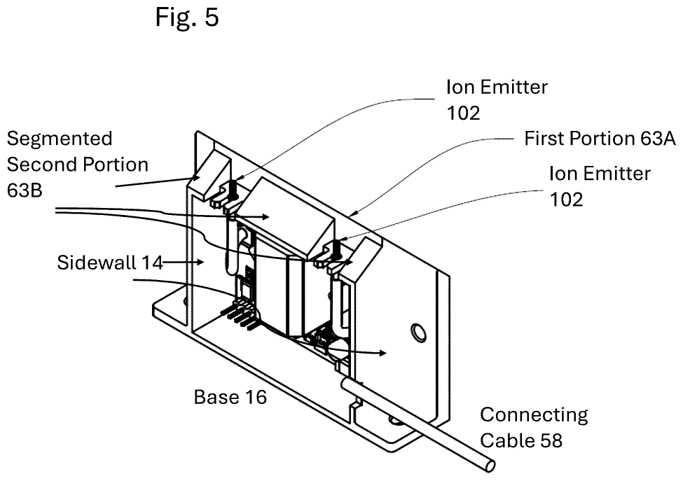

FIG. 5 illustrates an isometric view of the ion generator device shown in FIG. 1 without the cover in accordance with aspects of the disclosure;

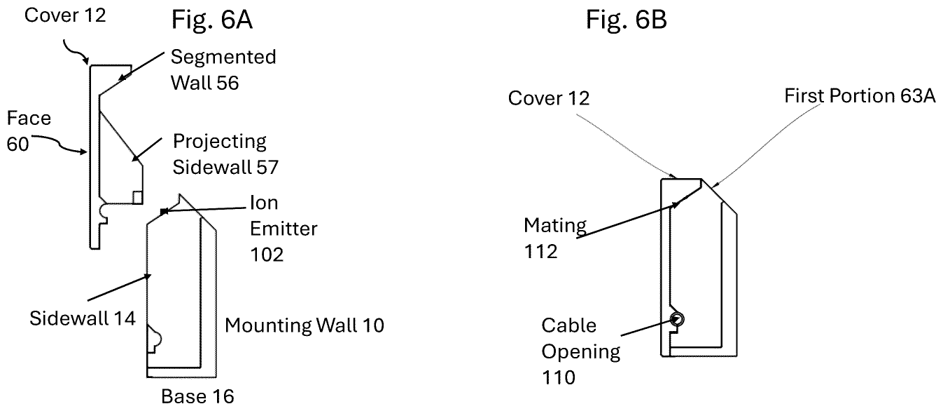

FIG. 6A illustrates a side view of the ion generator device shown in FIG. 1 with the cover separated and FIG. 6B illustrates a side view of the ion generator device shown in FIG. 1 with the cover attached in accordance with aspects of the disclosure;

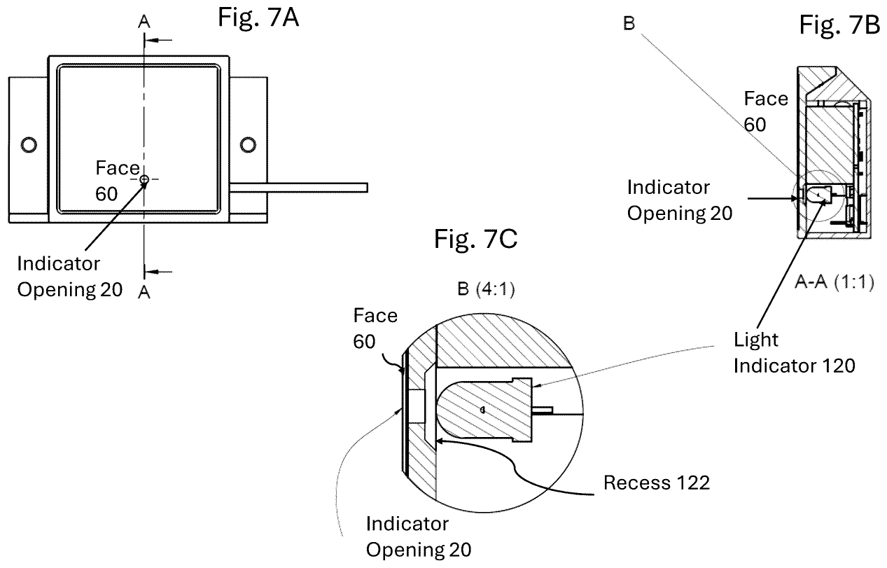

FIG. 7A illustrates a front view of the ion generator device shown in FIG. 1 in accordance with aspects of the disclosure;

FIG. 7B illustrates a sectional view along the A-A line in FIG. 7A showing the light indicator and indicator opening;

FIG. 7C illustrates a zoomed view of the area B in FIG. 7B;

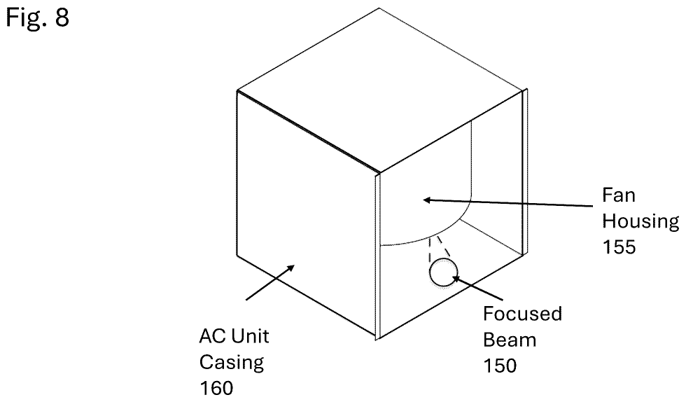

FIG. 8 illustrates the ion generator device shown in FIG. 1 in an example installation location, projecting a focused beam on an external surface in accordance with aspects of the disclosure;

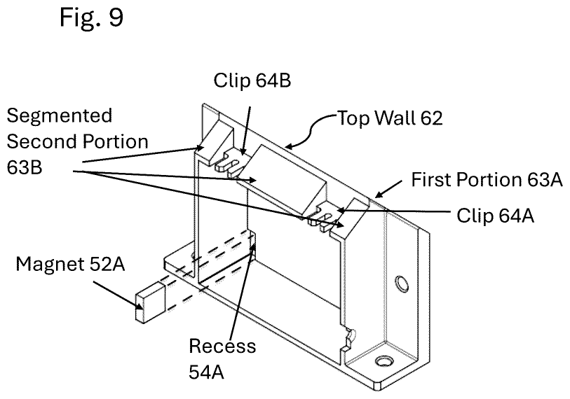

FIG. 9 illustrates an exploded view of a portion of another ion generator device in accordance with aspects of the disclosure;

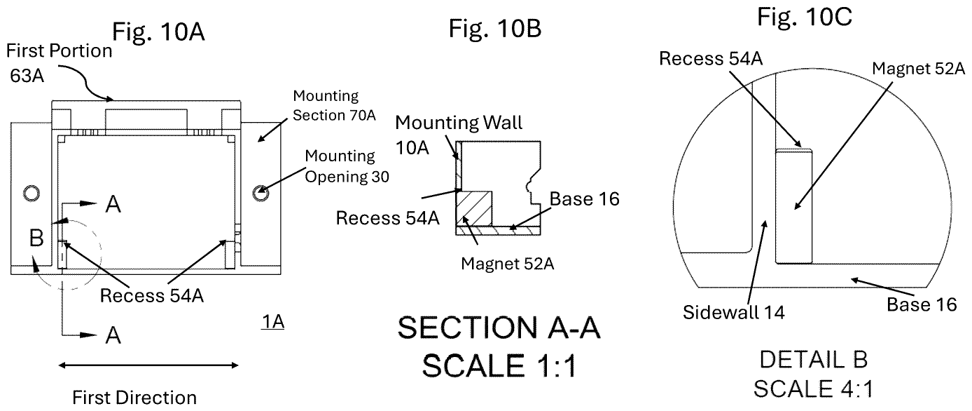

FIG. 10A illustrates a view of the another ion generator device in accordance with aspects of the disclosure;

FIG. 10B illustrates a sectional view along the A-A line in FIG. 10A; and

FIG. 10C illustrates a zoomed view of the area B in FIG. 10A.

Aspects of the disclosure provide an ion generator device 1 which is capable of being mounted using two different methods. For example, the ion generator device 1 has a mounting wall 10 which is configured to be mounted using the two different methods. This enables the ion generator device 1 to be mounted in various conduits and to different surfaces.

Aspects of the disclosure provide an ion generator device 1 which the status of the ion generator device 1 is readily viewable outside of the ion generator device 1 such as from a distance. For example, a focused beam 150 is projected through an indicator opening 20 in one of the walls of the ion generator device 1. The focused beam 150 can be viewed on an external surface.

Aspects of the disclosure provide an ion generator device 1 which produces application specific target ion counts for various target applications while meeting standards for ozone production.

FIG. 1 illustrates an isometric view of an ion generator device 1 in accordance with aspects of the disclosure. The ion generator device 1 may comprise a mounting wall 10 such as described above, a pair of sidewalls 14, a base 16, a top wall 62 (shown in FIG. 2) and a cover 12. The mounting wall 10, the pair of sidewalls 14, the base 16 and top wall 62 may be integrally formed. In some aspects of the disclosure, the mounting wall 10, the pair of sidewalls 14, the base 16 and top wall 62 may be fabricated via additive manufacturing such as via 3D printing. The sidewalls 14 and base 16 project from the mounting wall 10.

At least a portion of the mounting wall, the pair of sidewalls 14, the base 16, and the top wall 62 form a compartment to hold a circuit board 50 having components for regulating the production of ions.

In an aspect of the disclosure, the pair of sidewalls 14 project from a position of the mounting wall 10 offset in a first direction of the respective edges of the mounting wall. This provides two mounting sections 70A, 70B of the mounting wall 10 configured for mounting using a first mounting method. FIG. 3A shows the mounting sections 70A, 70B. The mounting sections 70A, 70B have mounting openings 30 configured to receive mounting hardware for mounting the ion generator device 1 to an external surface.

However, in other aspects of the disclosure, the pair of sidewalls 14 may project from the edges of the mounting wall 10 in the first direction.

The mounting wall 10 also has two recesses 54. Each recess 54 (also referred to herein as magnet pocket) is dimensioned to receive a magnet 52. In some aspects, the magnet pocket may be dimensioned to hold the entire magnet 52. In other aspects, as described later, the magnet pocket (in the mounting wall) may be dimensioned such that part of a magnet is external to the magnet pocket. In an aspect of the disclosure, each recess 54 may be oversized to facilitate easy insertion of each magnet 52 (such as in the longitudinal and transverse direction of the magnet (first direction and second direction). As shown in FIG. 3A (cover 12 is removed in FIG. 3A), a portion of the recess may be within a corresponding mounting section 70A or 70B and another portion of the recess may be between the pair of sidewalls 14 in the first direction. However, the location of each recess 54 is not limited to this position. For example, each recess 54 may be entirely between the opposing sidewalls 14. Additionally, although the figures show the recesses 54 aligned in the second direction, the recesses 54 need not be aligned.

In an aspect of the disclosure, the edges of each recess 54 may be chamfered (chamfer edge 100). The chamfer edge 100 also facilitates easy insertion and enables the magnets 52 to be self-centered. In a case where the recesses are located as shown in FIG. 3A, a magnet 52 may be inserted into a recess 54 from the compartment-side (e.g., between the pair of sidewalls 14 in the first direction) and slide toward a respective edge of the mounting wall 10 in the first direction until the magnet 52 reaches the end of the recess 54. Once positioned, the oversized space may be filled with epoxy to secure the magnet 52 in place. The chamfer edge 100 also provides a volume between the sides of the magnets and the mounting wall 10 for the epoxy. Other filling materials may be used.

A magnet 52 may be secured in position in other manners. For example, there may be a pressure fit between a magnet 52 and a recess 54. In other aspects, once a circuit board 50 is positioned within the compartment and secured, the circuit board 50 and edges of each recess 54 may prevent movement.

FIG. 3B illustrates a cross-section showing the recess 54. The cross-section is taken along line A-A in FIG. 3A, which is through the compartment (and not the magnet 52). FIG. 3D illustrates another cross-section. In FIG. 3D, the cross-section cuts through the magnet 52.

In some aspects of the disclosure, the mounting wall 10 may have different thicknesses in the third direction. For example, the mounting wall 10 may be thicker in the mounting sections 70A, 70B, than in the middle, e.g., between the sidewalls 14. The different thicknesses are shown in FIG. 3B and FIG. 3D. For example, in a case where a portion of the magnet 52 is within the mounting section 70A, 70B, respectively, the mounting wall 10 may be thicker to cover the magnet 52 such as shown in FIG. 3E (zoomed view of FIG. 3D).

The magnetic field at an outer mounting surface (e.g., the interface between the ion generator device 1 and an external surface where the ion generator device 1 is mounted to) is a function of the distance D between the magnet 52 and the outer mounting surface: the distance impacts the magnetic loss. The distance D is shown in FIG. 3C (which is a zoomed view of FIG. 3B). This distance D is set such that the magnetic field at the interface is sufficient to hold the ion generator device 1 to an external surface. The distance D may be based on the type of magnet 52 used and the material of the external surface. The thicker this distance D is, the larger the magnetic loss is. While a thinner distance D minimizes the magnetic losses, a thinner wall section may be more difficult to manufacture. In an aspect of the disclosure, the distance D may be between 0.2 mm and 0.6 mm. However, depending on the method of manufacturing, the distance D may be less than 0.2 mm. In other aspects, the distance may be larger.

In other aspects, in a case where each magnet 52 is aligned with the compartment, the mounting wall 10 may have the same thickness in the first direction.

In other aspects of the disclosure, the outer surface/interface of the mounting wall may have a pair of cutouts aligned with the recesses 54. In this aspect, each magnet 52 may be inserted into a recess 54 from the outer surface/interface side. The ion generator device 1 may also comprise two magnet covers insertable into the cutouts, respectively, to cover a respective magnet 52. The magnet cover acts as an access panel. The magnet cover may be attached to the ion generator device 1 is any suitable manner. In some aspects, the magnet cover may be attached via epoxy.

In other aspects, each recess 54 may be formed from the outer surface/interface and a portion of the mounting wall 10 may be between each magnet 52 other parts of the ion generator device 1 in the third direction. For example, a portion of the mounting wall 10 may be a physical barrier between each magnet 52 and the circuit board 50 in the third direction.

In an aspect of the disclosure, the base 16 extends the entire length of the ion generator device 1 in the first direction. In this case, optionally, the base 16 may also have a mounting opening(s) 30. The mounting opening(s) 30 in the base 16 may align with the mounting opening(s) in the mounting wall 10 (respectively) in the first direction. However, the mounting openings need not be aligned. Both sets of mounting openings 30 may be used in a case where the ion generator device 1 is mounted to a structure having two generally orthogonal surfaces.

In an aspect of the disclosure, the top wall 62 may comprise a first portion 63A and a second portion (segmented second portion 63B). The first portion 63A may extend (project) from the mounting wall 10. In an aspect of the disclosure, the first portion 63A may extend from the mounting wall 10 in a non-orthogonal angle towards the cover 12 (when attached).

In an aspect of the disclosure, the angle may be based on the manufacturing process. For example, in additive manufacturing, such as 3D printing, angled printing reduces the printing time and cost. This is because the angled printing reduces or eliminates any overhang during the printing process. An overhang would require the use of support material which increases the printing time and cost for the support material and manual labor to add/remove the support material. Additionally, the angled printing enables auto-ejection of the part from a printing plate. During the additive manufacturing process, the first portion 63A of the top wall 62 would be manufactured first. The top of the mounting wall 10 may have the same angle and the first portion 63A.

In other aspects, the ion generator device 1 may be manufactured using injection molding.

In other aspects, the first portion 63A may project orthogonally from the mounting wall 10 toward the cover.

In an aspect of the disclosure, the first portion 63A extends between the pair of sidewalls 14 in the first direction.

In an aspect of the disclosure, the second portion may be segmented (segmented second portion 63B). For example, the segmented second portion 63B may comprise a plurality of segments. The segments may project from the first portion 63A. The segments may also project from the mounting wall 10. In some aspects, the segments may be angled with respect to the first portion 63A such as being angled downward toward the base 16. Each segment may also be offset from the top edge of the first portion 63A in the second direction. This offset allows for clearance for the cover 12 to be attached.

In an aspect of the disclosure, a segment may be hollowed out as opposed to filled as shown in the figures.

In an aspect of the disclosure there is a gap(s) between the segments. FIG. 2 illustrates two gaps. In some aspect, clips 64A, 64B (or other means for holding the ion emitters 102) may be positioned in the gaps, respectively. The clips 64A, 64B are configured to hold the ion emitters 102, respectively such as shown in FIGS. 4 and 5. In an aspect of the disclosure, the clips 64A, 64B may center each ion emitter 102 with in a respective gap. Centering the ion emitter 102 with respect to a gap maximizes the ion output efficiency of the ion emitter 102. If the ion emitter 102 gets too close to a side of the gap (and a segment), the efficiency is reduced. The efficiency may be reduced due to a short to a wall.

In an aspect of the disclosure, the clips 64A, 64B may be aligned in the second direction with the bottom of the segment second portion. The position of the clips 64A, 64B may depend on the type of ion emitter 102 and length.

The clips 64A, 64B may be positioned to maintain a target distance in the first direction between the ion emitters 102.

The pair of sidewalls 14 may extend in the second direction between the top wall 62 and the base 16. In an aspect of the disclosure, one of the sidewalls may have a notch dimensioned to receive the connecting cable 58 which connects the circuit board 50 to an external power source (not shown). However, in other aspects, another wall of the ion generator device 1 may have the notch.

The cover 12 may be attached to cover the compartment. When attached, the cover 12 extends between the pair of sidewalls 14 in the first direction and the base 16 and top wall 62 in the second direction. The cover 12 may comprise a face 60, a segmented wall 56 and projecting sidewall(s) 57 as shown in FIG. 2 (one projecting sidewall 57 is visible).

The segmented wall 56 may comprise a plurality of segments. In an aspect of the disclosure, the number of segments in the segmented wall 56 may equal the number of segments in the segmented second portion 63B. Additionally, the segments in the segmented wall 56 may align in the first direction with the segments in the segmented second portion 63B.

As shown in FIGS. 2 and 6A, the interior surface of a segment of the segmented wall 56 may be angled upward. The upward angle may correspond to the downward angle of the interior surface of a segment of the segmented second portion 63B. When the cover 12 is attached, the corresponding angles, enable the corresponding segments to mesh or mate 112 as shown in FIG. 6B.

The cover 12 may have a pair of projecting sidewall(s) 57 adjacent the edges of the cover 12 in the first direction. When the cover 12 is mounted, the projecting sidewall(s) 57 may be positioned within the compartment into an epoxy fill to secure the cover 12 to the housing. In other aspects, the cover 12 may be mounted using a latches or other mounting means. In this aspect of the disclosure, the projecting sidewall(s) 57 may be omitted.

In some aspects, the cover 12 may also comprise a corresponding notch to the notch in a sidewall to receive the connected cable 58 which connects the circuit board 50 to an external power source (not shown), i.e., to complete the cable opening 110. In some aspects, the corresponding notch may be on the projecting sidewall 57.

In some aspects, the projecting sidewall 57 may have a cutout to provide a space for certain circuit components which may be attached to the circuit board 50.

In some aspects, one of the walls of the ion generator device 1 may have an indicator opening 20. While the figures illustrate the indicator opening 20 on the cover 12, for descriptive purposes, the indicator opening 20 is not limited to be on the cover 12. As depicted, such as in FIG. 2, the face 60 of the cover 12 has an indicator opening 20. The indicator opening 20 is dimensioned to pass a focused beam 150 to the outside of the ion generator device 1 which is emitted by a light indicator 120. In some aspects, the indicator opening 20 may have a film or lens covering the opening. The film may be transparent to the wavelength of light emitted by the light indicator 120. The lens may be configured to further focus the focused beam 150. The transparent film or lens may be used to prevent debris (including moisture) from entering the ion generator device 1.

The position of the indicator opening, e.g., which wall has the same, may be based on the application, such as the installation location (including allotted space and installation orientation).

In an aspect of the disclosure, the ion generator device 1 may receive either DC or AC. The ion generator device 1 may be configured to receive a first input or receive a second input. In an aspect of the disclosure, the first input may be about 12-24V AC or DC. The first input may also be referred to herein as a low voltage input. The second input may be about 85 VAC to about 265 VAC. The second input may also be referred to herein as a line voltage input.

The circuit board 50 may include and/or be attached to circuit components which are configured to receive either the first input or second input, rectify the same and convert to a regulated predetermined VDC. Additionally, the circuit board 50 may include and/or be attached to circuit components configured to receive the regulated predetermined VDC and provide a voltage for the ion emitters 102. The rectifier may be a full wave rectifier or a half wave rectifier. For example, for the low voltage input, a full wave rectifier may be used whereas for the line voltage input, a half wave rectifier may be used.

The circuit board 50 may also include a programable high voltage (HV) generator. In some aspects, the programmable HV generator may include firmware for setting the HV output voltage. For example, the HV output voltage may be from 0 to 10 KV DC for the positive ion emitter and −0 to −10 KV DC for the negative ion emitter. The output range described herein is for descriptive purposes only and other ranges may be used. The programmable HV generator may also comprise a transformer.

The circuit board 50 may also comprise one or more relay dry contacts. In some aspects, one of the relay dry contacts may be electrically connected to a Building Management System (BMS).

The light indicator 120 may be mounted to the circuit board 50 and project therefrom. The light indicator 120 may be aligned with the indicator opening 20. For example, in a case where the indicator opening is in the cover 12, the light indicator 120 may be aligned in the first direction and the second direction, such that it is viewable from the third direction. FIG. 7B is a cross-section showing an example alignment of the light indicator 120 and the indicator opening 20. The cross-section is taken along the line A-A in FIG. 7A (which is through the center of the indicator opening 20). The light indicator 120 is adjacent to the indicator opening 20.

However, in other aspects, the indicator opening 20 may be in one of the sidewalls 14, the base 16 or the top wall 62. When the indicator opening 20 is located in any of these walls, the light indicator 120 may be mounted within the ion generator device 1 in a different orientation such that the focused beam 150 is viewable from the outside of the ion generator device 1. In some aspects, the light indicator may be electrically connected to the circuit board and positioned on a wall of the ion generator device 1.

In other aspects of the disclosure, there may be more than one indicator opening 20 and light indicator 120. Each light indicator 120 may indicate a different status of the ion generator device 1.

As shown in FIG. 7C (which is a zoomed view of area B in FIG. 7B), the interior surface of the face 60 of the cover 12 may have a recess 122 around the indicator opening 20. The recess 122 minimizes the distance between the tip of the light indicator 120 and the indicator opening 20. Additionally, the recess helps focus the emitted beam into the indicator opening 20. Similarly, when another wall of the ion generator device 1 has the indicator opening 20, the wall may also have the recess 122. In some aspects, the recess 122 may be formed by a bevel cut in the surface surrounding the indicator opening 20.

In some aspects of the disclosure, the shape of the indicator opening 20 may be circular. To focus the light emitted from the light indicator 120 (focused beam 150), the diameter of the indicator opening 20 may be small such as on the order of 3 mm. In other aspects, the size of the indicator opening 20 may be a function of the size of the light indicator 120. For example, an LED may range in size from about 5 mm to less than 1 mm. In a case where a 5 mm LED is used, the indicator opening 20 may match the size such as being about 5 mm. In a case where a smaller LED is used such as a 1.8 mm, the indicator opening 20 may be about 1.8 mm.

Additionally, to create a tight beam of light, the light indicator 120 may have a narrow viewing angle. The viewing angle may be less than a predetermined angle. For example, the viewing angle may be about 30°. The viewing angle described herein is descriptive purposes only and other ranges may be used. In some aspects of the disclosure, the light indicator 120 may be a light emitting diode (LED). Where more than one light indicator 120 is included, each light indicator 120 may be a different color.

The light indicator 120 is a status indicator to indicate the operating status of the ion generator device 1. The combination of the light indicator 120 and the indicator opening 20 provides the focused beam 150 which may be projected onto adjacent surfaces at the installation site. This enables the installer or operator to know whether the ion generator device 1 is working after installation. The focused beam 150 provides a clear visual indication even when the ion generator device 1 itself is not visible to the operator or installer due to the location of the mount such as shown in FIG. 8. In FIG. 8, the ion generator device 1 is mounted on a bottom surface of the fan housing 155. The fan housing 155 is surrounded by an AC unit casing 160. Given the proximity of the AC unit casing 160 to the bottom surface of the fan housing 155, it is difficult to see the ion generator device 1 itself. However, since the ion generator device 1 produces the focused beam 150 which is visible at a distance from the ion generator device 1, the status of the ion generator device can be known. The focused beam 150 is shown illuminated on the AC unit casing 160 in FIG. 8 (an example of an external surface).

The combination of gaps between the segments of the segmented wall 56 of the cover 12 and segmented second portion 63B, define emitter pockets 24A and 24B. The emitter pockets 24A, 24B are dimensioned such that the tip of the ion emitters 102 are recessed with respect to a top surface of the ion generator device 1 and held by the clips 64A, 64B as described above. This combination enables the pressure around the ion emitters 102 to be lower than if the ion emitters 102 are external (e.g., above the top surface). The pressure is reduced because the ion emitters 102 are exposed to less air molecules and slower airflow. The lower air molecule count increases the mean free path for the electrons because there are less molecules to collide with, which allows for the electrons to gain a higher kinetic energy between the collisions, resulting in a lower threshold for ionization. The reduced number of collisions also means that electrons can be accelerated to high energies by a particular electric field more easily, which also enhances the ionization process. In other words, the emitter pockets 24A, 24B allow the production of a target number of ions while using a lower output (voltage) from the programmable HV generator (which in turn reduces the electric field strength between the ion emitters 102). By reducing the electric field strength between the ion emitters 102 (and providing a dielectric barrier as will be described later), the creation of ozone through corona discharge is reduced or eliminated.

Ozone (O₃) is formed through a recombination of oxygen atoms (O) with oxygen molecules (O₂). But, since there are less air molecules, the rate of a three-body collision, which is necessary for the formation of ozone, is lower. For example, the three body collisions are notated as follows: O+O₂+M→O₃+M, where M is the third body. This leads to a reduction of ozone production efficiency since the needed collisions are less frequent.

A segment of the segmented second portion 63B and the corresponding segment of the segmented wall 56 which is between the ion emitters 102 in the first direction provide a physical barrier between the ion emitters 102 (collectively an example of “a wall” and where a portion of the wall may be formed from a portion of the top wall 62). The physical barrier does not allow air to have a direct path between the ion emitters 102 where the electric field would be the strongest. This reduces the production of ozone since, at the operating voltages, the strong electric field would cause a dielectric breakdown (of any dielectric property of air). Air has a relatively low dielectric strength.

In another aspect the disclosure, these segments (between the ion emitters 102) may be made of a dielectric material. In some aspects, these segments may be formed of a material such as acrylonitrile butadiene styrene (ABS), polyethylene (PE), polycarbonate (PC), polypropylene (PP), polyethylene terephthalate (PET), polyvinyl chloride (PVC), and/or polytetrafluoroethylene (PTFE). These segments may also be formed from an acrylic (plexiglass), In other aspects, these segments may be formed from fiberglass. The dielectric strength of these materials is higher than air and at the operating voltages will not break down into conduction.

Depending on the application, other materials may be used including a glass such as fused silica.

In some aspects of the disclosure, all of the walls of the ion generator device 1 may be made of the same material as these segments for ease in manufacturing. However, different materials may be used for portions of the ion generator device 1 other than the segments between the ion emitter 102.

In other aspects of the disclosure, given that many applications require that the ion generator device 1 to be positioned in a small space, the ion generator device 1 may be relatively thin in the third direction. For example, in some aspects, the ion generator device 1 may be less than 2 in, in the third direction. In other aspects, the ion generator device 1 may be less than 1.5 in, in the third direction. In other aspects, the ion generator device 1 may be less than 1 in, in the third direction. In this aspect, the ion emitters 102 may be midway in the third direction, e.g., 0.5 in.

Comparative example of ion and ozone generation with and without emitter pockets 24A, 24B: With the ion emitters 102 removed from the Clips 64A and 64B, the ion emitters 102 were allowed to protrude perpendicular to circuit board 50 (or, in the Third Direction). This exposed the ion emitters 102 to ambient air. In this orientation, ion output was measured using an Alpha Labs model AIC2 ion counter at a distance of 1 inch from each ion emitter. Ozone output was also measured. After installing the ion emitters 102 into the emitter pockets 24A, 24B, the same tests were rerun under the same conditions using the same equipment. While an increase in ion output and decrease in ozone output were expected, the ion output almost doubled while the ozone production was reduced by about half.

The ion generator device 1 may be incorporated into a heating, ventilating, and air-conditioning system (HVAC) such as into an air conduit thereof. The air conduit may be any structure forming an enclosed air channel for air to flow. For example, the air conduit may be a structure such as, but not limited to an air handling unit (AHU), an air duct, a rooftop unit (RTU), a dedicated outdoor air system (DOAS), packaged Terminal Air Conditioners (PTAC) or a makeup air unit (MAU). The air conduit may also be a fan coil unit (FCU). The air conduit may also be a wall mount unit or a ceiling cassette.

As described above, the magnet(s) 52 are positioned with the mounting wall 10 and within the recess(es) 54 such that the longitudinal axis of the magnet(s) is generally parallel to the longitudinal axis of the mounting wall 10 (and in turn) a mounting surface of the target. However, in other aspects of the disclosure, the magnet(s) 52A may be positioned in a different orientation. For example, the magnet(s) 52A may be positioned such that the longitudinal axis of the magnet(s) 52A is generally orthogonal to the longitudinal axis of the mounting wall 10A (and in turn) a mounting surface of the target. FIG. 9 illustrates an exploded view of a portion of an ion generator device 1A with a magnet 52A in this orientation. FIG. 9 illustrates one magnet 52A for illustrative purposes, however, the ion generator device 1A in accordance with this aspect may have multiple magnets 52A.

FIG. 10A illustrates the ion generator device 1A with the cover 12 removed. In this example, the recesses 54A are aligned with the component as viewed from the third direction. Compared to recess 54, recess 54A may be narrower in the first direction than recess 54. Similar to above, each recess 54A may also be oversized in the first direction and the second direction and have chamfered edges to facilitate insertion of the magnet 52A such as shown in FIG. 10C. Each recess 54A may be dimensioned in the third direction such that the distance D between a magnet 52A and the outer mounting surface is the same as above. In this aspect of the disclosure, only part of each magnet 52A may be inserted into a respective recess 54A. A portion of the magnet 52A is positioned within the compartment such as shown in FIG. 10B. As shown in FIG. 9, a magnet 52A may be placed into positioned without a need to slide in the first direction. Once positioned, the oversized space may be filled with epoxy to secure each magnet 52A in place. The chamfer edge also provides a volume between the sides of the magnets and the mounting wall 10A for the epoxy. Other filling materials may be used.

In some aspects, a respective magnet 52A may be in contact with a respective sidewall 14 which also helps keep the magnet 52A in position (such as shown in FIG. 10C). Additionally, in some aspects, a respective magnet 52A may be in contact with the base 16 which also helps keep the magnet 52A in position (such as shown in FIG. 10C). In an aspect of the disclosure, the ion generator device 1A may be manufactured using ejection molding or additive manufacturing. By having the magnets 52A oriented as shown in FIG. 10B supports manufacturing using ejection molding.

In other aspects of the disclosure, each magnet 52A may be framed on all sides. For example, projections from the mounting wall 10A may surround any exposed sides of a magnet 52A. This may reduce any magnetic interference on any electrical component on the circuit board 50 due to the magnetic field from the magnet 52A.

In an aspect of the disclosure, the ion generator device 1 may be mounted in a room such as mounted to any magnet surface such as ovens, refrigerator, dishwashers, etc.

In the discussion and claims herein, the term “about” indicates that the value listed may be somewhat altered, as long as the alteration does not result in nonconformance of the process or device. For example, for some elements the term “about” can refer to a variation of ±0.1%, for other elements, the term “about” can refer to a variation of ±1% or ±10%, or any point therein. For example, the term about when used for a measurement in mm, may include +/0.1, 0.2, 0.3, etc., where the difference between the stated number may be larger when the stated number is larger. For example, about 1.5 may include 1.2-1.8, where about 20, may include 19.0-21.0.

As used herein, the term “substantially”, or “substantial”, is equally applicable when used in a negative connotation to refer to the complete or near complete such a lack of an action, characteristic, property, state, structure, item, or result. For example, a surface that is “substantially” flat would either be completely flat, or so nearly flat that the effect would be the same as if it were completely flat. “Substantially” when referring to a shape or size may account for manufacturing tolerances where a perfect shapes, edges, turns, such as circular or sizes or angles may be difficult to manufacture.

References in the specification to “one aspect”, “certain aspects”, “some aspects” or “an aspect”, indicate that the aspect(s) described may include a particular feature or characteristic, but every aspect may not necessarily include the particular feature, structure, or characteristic. Moreover, such phrases are not necessarily referring to the same aspect. Further, when a particular feature, structure, or characteristic is described in connection with an aspect, it is submitted that it is within the knowledge of one skilled in the art to affect such feature, structure, or characteristic in connection with other aspects whether or not explicitly described.

The described aspects and examples of the present disclosure are intended to be illustrative rather than restrictive, and are not intended to represent every aspect or example of the present disclosure. While the fundamental novel features of the disclosure as applied to various specific aspects thereof have been shown, described and pointed out, it will also be understood that various omissions, substitutions and changes in the form and details of the devices illustrated and in their operation, may be made by those skilled in the art without departing from the spirit of the disclosure. For example, it is expressly intended that all combinations of those elements and/or method steps which perform substantially the same function in substantially the same way to achieve the same results are within the scope of the disclosure. Moreover, it should be recognized that structures and/or elements and/or method steps shown and/or described in connection with any disclosed form or aspects of the disclosure may be incorporated in any other disclosed or described or suggested form or aspects as a general matter of design choice. Further, various modifications and variations can be made without departing from the spirit or scope of the disclosure as set forth in the following claims both literally and in equivalents recognized in law.

What is claimed is:

1. An ion generator device comprising:
a mounting wall;
opposing sidewalls projecting from the mounting wall;
a first wall projecting from an edge of the mounting wall, the edge being an edge in a second direction orthogonal to a first direction, where the opposing sidewalls are connected to the first wall;
a second wall extending between the opposing sidewalls in the first direction, at least a portion of the mounting wall, the opposing sidewalls, the first wall and the second wall forming a compartment for a circuit board of the ion generator device, the circuit board being connected to a first ionizing element and a second ionizing element configured to produce ions, the first ionizing element and the second ionizing element being mounted in respective emitter pockets;
a cover extending between the opposing sidewalls in the first direction, the cover being configured to cover the compartment; and
a wall extending between the first ionizing element and the second ionizing element, wherein the wall is formed of a dielectric material and has a dielectric strength sufficient to maintain its dielectric property at operating voltages.

2. The ion generator device of claim 1, wherein the first ionizing element and the second ionizing element are mounted in the respective emitter pockets such that tips of the first ionizing element and the second ionizing element do not extend beyond the ion generator device.

3. The ion generator device of claim 2, further comprising a first clip and a second clip configured to respectively hold the first ionizing element and the second ionizing element such that the first ionizing element and the second ionizing element are held in a middle of a respective emitter pocket.

4. The ion generator device of claim 1, wherein the opposing sidewalls being offset from a first edge and a second edge of the mounting wall in a first direction and wherein the mounting wall has a first recess and a second recess, where the first recess and the second recess are configured to respectively receive a magnet for mounting the ion generator device to an external surface.

5. The ion generator device of claim 4, wherein the first recess extends both on a first side of one of the opposing sidewalls and a second side of the one of the opposing sidewalls in the first direction and the second recess extends both on a first side of another opposing sidewall and a second side of the another opposing sidewall in the first direction.

6. The ion generator device of claim 4,
wherein the first recess and the second recess are dimensioned to enable a magnetic field of a respective magnet to be a sufficient strength at the external surface to hold the ion generator device to the external surface,
wherein edges of the first recess and the second recess are chamfered, and
wherein the first recess and the second recess extend in the first direction a greater distance than the respective magnet.

7. The ion generator device of claim 4, wherein the mounting wall has at least two openings configured to receive mounting hardware to attach the ion generator device to the external surface, where the at least two openings are adjacent to the first edge and the second edge, respectively and/or wherein the first wall extends from the first edge to the second edge in the first direction and the first wall has at least two openings configured to receive mounting hardware to attach the ion generator device to another external surface.

8. The ion generator device of claim 1, further comprising an ionization indicator light and a wall of the ion generator device has an indicator opening aligned with the ionization indicator light.

9. The ion generator device of claim 8, wherein an interior surface of said wall with the indicator opening has a bevel edge around the indicator opening, wherein the ionization indicator light is positioned within the bevel edge adjacent to the indicator opening.

10. The ion generator device of claim 1, wherein the first ionizing element and the second ionizing element are ionizing needles or ionizing brushes.

11. The ion generator device of claim 1, wherein the first ionizing element produces positive ions and the second ionizing element produces negative ions or wherein the first ionizing element produces both positive and negative ions and the second ionizing element produces both positive and negative ions.

12. The ion generator device of claim 1, wherein a length of the ion generator device in a third direction orthogonal to the first direction and the second direction is equal to or less than about 1 in.

13. An ion generator device comprising:
a mounting wall;
opposing sidewalls projecting from the mounting wall, the opposing sidewalls being offset from a first edge and a second edge of the mounting wall in a first direction;
a first wall projecting from a third edge of the mounting wall, the third edge being an edge in a second direction orthogonal to the first direction, the opposing sidewalls are connected to the first wall;
a second wall extending between the opposing sidewalls, a portion of the mounting wall, the opposing sidewalls, the first wall and the second wall forming a compartment for a circuit board of the ion generator device, the circuit board being connected to a first ionizing element and a second ionizing element configured to produce ions; and
a cover extending between the opposing sidewalls, the cover configured to cover the compartment when attached,
wherein the mounting wall has a first recess and a second recess, where the first recess and the second recess are configured to respectively receive a magnet for mounting the ion generator device to an external surface.

14. The ion generator device of claim 13, wherein the first recess extends both on a first side of one of the opposing sidewalls and a second side of the one of the opposing sidewalls in the first direction and the second recess extends both on a first side of another opposing sidewall and a second side of the another opposing sidewall in the first direction.

15. The ion generator device of claim 13, wherein the first recess and the second recess are dimensioned to enable a magnetic field of a respective magnet to be a sufficient strength at the external surface to hold the ion generator device to the external surface.

16. The ion generator device of claim 13, wherein edges of the first recess and the second recess are chamfered and the first recess and the second recess extend in the first direction a greater distance than a respective magnet.

17. The ion generator device of claim 13, wherein the mounting wall has at least two openings configured to receive mounting hardware to attach the ion generator device to the external surface, where the at least two openings are adjacent to the first edge and the second edge, respectively and/or wherein the first wall extends from the first edge to the second edge in the first direction and the first wall has at least two openings configured to receive mounting hardware to attach the ion generator device to another external surface.

18. An ion generator device comprising:
a mounting wall;
opposing sidewalls projecting from the mounting wall;
a first wall projecting from an edge of the mounting wall, the edge being an edge in a second direction orthogonal to a first direction, where the opposing sidewalls are connected to the first wall;
a second wall extending between the opposing sidewalls in the first direction, at least a portion of the mounting wall, the opposing sidewalls, the first wall and the second wall forming a compartment for a circuit board of the ion generator device, the circuit board being connected to a first ionizing element and a second ionizing element configured to produce ions;
a cover extending between the opposing sidewalls, the cover configured to cover the compartment when attached;
an ionization indicator light; and
wherein a wall of the ion generator device has an indicator opening aligned with the ionization indicator light such that the ionization indicator light is viewable on an external surface.

19. The ion generator device of claim 18, wherein the ionization indicator light is a light emitting diode (LED) with a viewing angle of less than a predetermined angle.

20. The ion generator device of claim 18, wherein a protective film or lens is installed on or in the indicator opening.

21. The ion generator device of claim 18, wherein an interior surface of the wall has a bevel edge around the indicator opening, wherein the ionization indicator light is positioned within the bevel edge adjacent to the indicator opening.

22. The ion generator device of claim 18, wherein the indicator opening has a diameter of about 3 mm.
